(19) United States
(12) Patent Application Publication
(10) Pub. No.: US 2024/0226600 A9
(43) Pub. Date: Jul. 11, 2024

(54) METHOD AND DEVICE FOR TREATING RADIATION DERMATITIS

(71) Applicant: MUREVA PHOTOTHERAPY INC., Strongsville, OH (US)

(72) Inventors: Vedang KOTHARI, San Diego, CA (US); Jordan W. OJA, Stow, OH (US); Jason D. LAZZARA, Brooklyn Heights, OH (US); Samuel J. SHELNUTT, Olmsted Township, OH (US); Nancy LIPKO, Beachwood, OH (US)

(73) Assignee: MUREVA PHOTOTHERAPY INC., Strongsville, OH (US)

(21) Appl. No.: 18/546,341

(22) PCT Filed: Feb. 17, 2022
(86) PCT No.: PCT/US2022/016770

§ 371 Date: Aug. 14, 2023

Related U.S. Application Data
(60) Provisional application No. 63/150,633, filed on Feb. 18, 2021.

(51) Int. Cl. A61N 5/06

(52) U.S. Cl. CPC A61N 5/0616; A61N 2005/0626; A61N 2005/0645; A61N 2005/0652

(57) ABSTRACT

A phototherapy device is provided for treating a medical condition by delivering phototherapy using a pad and a light source supported by the pad. The light source includes multiple light emitters that are located at different positions on the pad and that emit treatment electromagnetic radiation. A treatment property of the phototherapy varies spatially based on medical properties of the anatomical treatment area. The medical properties include at least one of anatomical structures or the medical condition

### RELATED APPLICATIONS

This application claims the benefit of 63/150,633 filed on Feb. 18, 2021. Which is herein incorporated by reference in its entirety.

### TECHNICAL FIELD

The present disclosure relates generally to radiation dermatitis and more particularly to a method and system for treating and/or preventing radiation dermatitis.

### BACKGROUND

Acute skin reactions or toxicities are one of the most common side effects of radiation therapy and/or chemotherapy. Several radiotherapy-related factors may potentially affect its severity such as total dose, fractionation, radiation energy, volume of treated regions, treatment duration and treatment site. Additionally, patient-related factors such as age, comorbid conditions, skin phototype, and genetic predisposition influence skin toxicity. One distressing skin toxicity, radiation dermatitis, is the result of radiation's effect on the epithelium and underlying structures of the skin, and is characterized by erythema (painful redness), dry or moist desquamation, and ulceration.

Most patients with head, neck, or breast cancer that undergo radiotherapy will develop some grade of acute radiation dermatitis. Other patient populations with high incidences of radiation dermatitis include those with skin melanomas. Radiation dermatitis appears in the area that receives focused radiation. In head and neck cancer patients, the anterior neck and lower cheek areas see the highest radiation dosage. In breast cancer patients, the affected breast and nearest underarm see the highest radiation dosage. In both head, neck, and breast cancers, cancer is most likely to spread to the nearest lymph nodes (neck and axillary).

Beyond radiotherapy interventions for cancer treatment, fluoroscopically guided intervention procedures may induce acute skin toxicities when used at high doses, for long procedure times, and at frequent rates.

There are no universally accepted guidelines for treatment of acute skin toxicities like radiation dermatitis. Treatment and prevention vary considerably from practice to practice. The current standard of care includes frequent washing, use of specific deodorants, topical agents/creams, dressings, and topical antibiotics. However, several medications, topical agents, dressings, and radioprotectors have been proposed for the prevention and treatment of radiation dermatitis.

### SUMMARY

Radiation dermatitis, chemotherapy and radiation therapy toxicities, and anti-cancer treatment side effects can be treated and/or prevented using photobiomodulation. Photobiomodulation is the use of a specific dosage of light (e.g., wavelength, power, and time) to stimulate biological functions, including wound healing. Photobiomodulation may be performed using laser therapy, but laser therapy requires a trained clinician, can only treat small areas of tissue at a time, and each treatment can require 40 minutes per day.

The present disclosure provides a phototherapy device for treating a medical condition using a pad including light emitters located at different positions on the pad, such that a treatment property of the phototherapy varies spatially based on medical properties of the anatomical treatment area. For example, processor circuitry of the phototherapy device may separately control one or more of the light emitters such that the treatment property varies based on anatomical structures and/or the medical condition (e.g., location of irritation, etc.).

While a number of features are described herein with respect to embodiments of the invention; features described with respect to a given embodiment also may be employed in connection with other embodiments. The following description and the annexed drawings set forth certain illustrative embodiments of the invention. These embodiments are indicative, however, of but a few of the various ways in which the principles of the invention may be employed. Other objects, advantages, and novel features according to aspects of the invention will become apparent from the following detailed description when considered in conjunction with the drawings.

### BRIEF DESCRIPTION OF THE DRAWINGS

The annexed drawings, which are not necessarily to scale, show various aspects of the invention in which similar reference numerals are used to indicate the same or similar parts in the various views.

FIG. 1 is a schematic diagram of an exemplary embodiment of a phototherapy device providing electromagnetic radiation having a property that varies spatially based on medical properties of an anatomical treatment area.

FIG. 2 is a schematic diagram of an exemplary embodiment of the phototherapy device having two types of light emitters and light emitters non-uniformly spaced on a pad.

FIG. 3A is a schematic diagram of an exemplary embodiment of the phototherapy device having a first group of light emitters separately controllable from a second group of light emitters and including air passages.

FIG. 3B is a side view of the phototherapy device of FIG. 3A along line A.

FIG. 3C is a side view of an exemplary embodiment of the phototherapy device.

FIG. 4A is a schematic diagram of a side view of an exemplary embodiment of the phototherapy device for treating a throat of a patient.

FIG. 4B is a back view of the phototherapy device of FIG. 4A.

FIG. 5A is a side schematic diagram of an exemplary embodiment of the phototherapy device for treating a breast of the patient.

FIG. 5B is a back view of the phototherapy device of FIG. 5A.

FIG. 6 is a schematic diagram of an exemplary embodiment of the phototherapy device including slits.

FIG. 7 is a schematic diagram of an exemplary embodiment of the phototherapy device having multiple pads.

FIG. 8 is a schematic diagram of an exemplary embodiment of the phototherapy device for treating a throat of the user using light emitters located between sternocleidomastoid muscles of the patient.

FIG. 9A is a top schematic diagram of an exemplary embodiment of the phototherapy device having a pad including articulating structures.

FIG. 9B is a side schematic diagram of the phototherapy device of FIG. 9A.

FIG. 10 is a flow diagram of an exemplary embodiment of a method for treating a medical condition by applying phototherapy using the phototherapy device.

The present invention is described below in detail with reference to the drawings. In the drawings, each element with a reference number is similar to other elements with the same reference number independent of any letter designation following the reference number. In the text, a reference number with a specific letter designation following the reference number refers to the specific element with the number and letter designation and a reference number without a specific letter designation refers to all elements with the same reference number independent of any letter designation following the reference number in the drawings.

### DETAILED DESCRIPTION

According to a general embodiment, a phototherapy device is provided for treating a medical condition by delivering phototherapy using a pad and a light source supported by the pad. The light source includes multiple light emitters that are located at different positions on the pad and that emit treatment electromagnetic radiation. A treatment property of the phototherapy varies spatially based on medical properties of the anatomical treatment area.

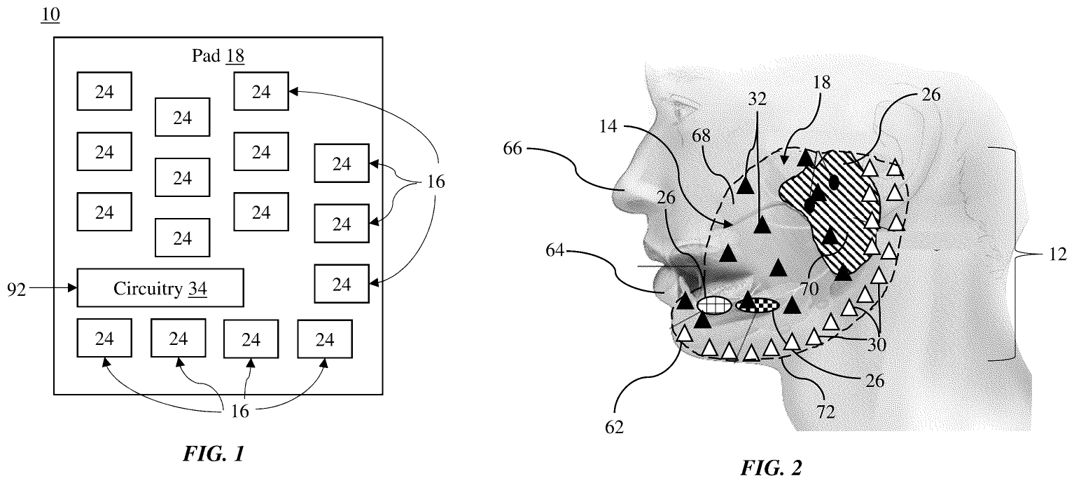

Turning to FIG. 1, an exemplary embodiment of a phototherapy device 10 is shown for treating a medical condition by delivering phototherapy to an anatomical treatment area 12 of a patient via a skin surface 14 of the patient. The phototherapy device 10 includes a light source 16 and a pad 18. The light source 16 emits electromagnetic radiation 20. The pad 18 supports the light source 16, such that positioning the pad 18 adjacent to the skin surface 14 causes the electromagnetic radiation 20 output by the light source 16 (1) to be received by the skin surface 14 as treatment electromagnetic radiation 22 and (2) to interact with the anatomical treatment area 12. The light source 16 includes multiple light emitters 24 located at different positions on the pad 18, such that a treatment property of the treatment electromagnetic radiation 22 varies spatially. For example, at least one of wavelength, optical intensity, or optical dose of the treatment electromagnetic radiation 22 may vary spatially. The treatment property varies spatially based on medical properties of the anatomical treatment area 12, such as a location of anatomical structures 26 or the medical condition.

In the embodiment shown in FIG. 2, the light emitters 24 include at least one first type of light emitter 30 and at least one second type of light emitter 32. The treatment property of the electromagnetic radiation 20 emitted by the first type of light emitter 30 differs from the treatment property of the electromagnetic radiation 20 emitted by the second type of light emitter 32. For example, the total number of light emitters or a position of the light emitters may vary between the first type of light emitter 30 and the second type of light emitter 32, such that the treatment property of the treatment electromagnetic radiation 22 varies spatially.

In one embodiment, the first type of light emitter 30 may emit a first wavelength range of electromagnetic radiation 20 and the second type of light emitter 32 may emit a second wavelength range of electromagnetic radiation 22 that is different from the first wavelength range. Because the number and/or position of the first type of light emitters varies from the number and/or position of the second type of light emitters, the wavelength range of the electromagnetic radiation emitted by the phototherapy device will vary spatially depending on the position of the first and second type of light emitters.

In one embodiment, the phototherapy device 10 includes processor circuitry 34 that controls the emission of the electromagnetic radiation 20 by the light source 16 by separately controlling the first type of light emitters 30 from the second type of light emitters 32. For example, the processor circuitry 34 may control the first type of light emitters 30 and the second type of light emitters 34 based on the medical condition being treated. As an example, if the first type of light emitter 30 emits electromagnetic radiation having a longer wavelength of light (i.e., that will penetrate deeper into tissue) and the second type of light emitter 32 emits electromagnetic radiation having a shorter wavelength of light (i.e., that will not penetrate as deeply into tissue), then the processor circuitry 34 may cause the first type of light emitter 30 to emit a greater optical dosage than the second type of light emitter 32 when treating a medical condition primarily affecting bone tissue. Alternatively, when treating a medical condition affecting the skin, the processor circuitry 34 may cause the second type of light emitter 32 to emit a greater optical dosage than the first type of light emitter 30.

In the embodiment shown in FIG. 2, a positioning of the light emitters 24 varies spatially, such that the optical dose of the treatment electromagnetic radiation 22 varies spatially. For example, the light emitters 24 may be concentrated in areas overlapping the location of anatomical structures 26 when the pad 18 is placed in a defined location on the patient. In FIG. 2, the light emitters 24 are concentrated to overlap with the jawbone and at least one of the parotid, submandibular, or sublingual gland of the patient when positioned on the patient's face.

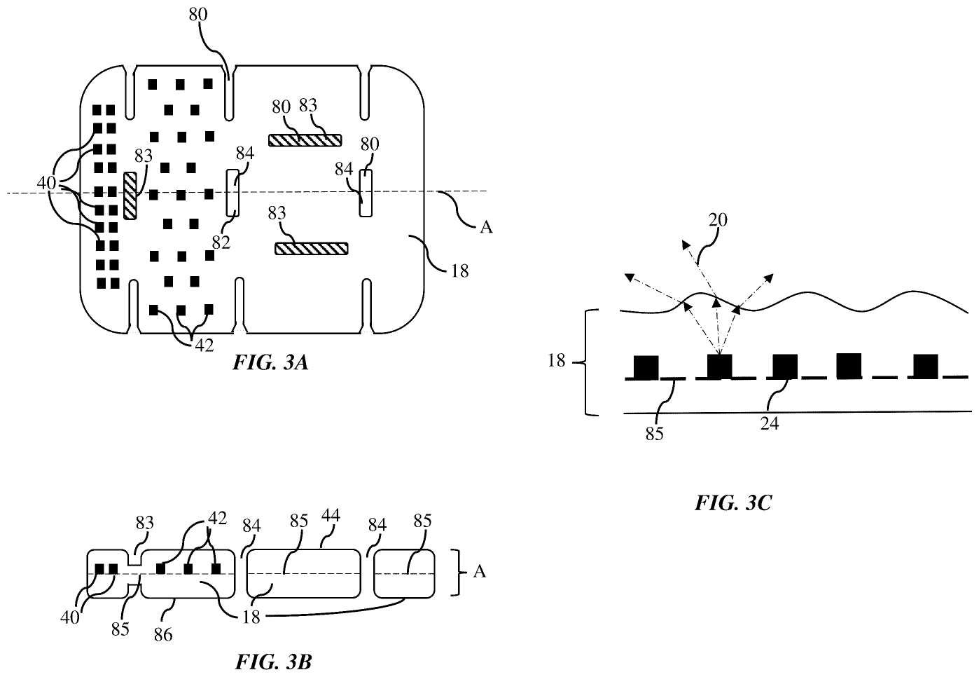

In the embodiment shown in FIG. 3, the light emitters 24 include at least a first group of light emitters 40 and a second group of light emitters 42. The electromagnetic radiation emitted by the first group of light emitters 40 may have different or the same properties (e.g., wavelength range, intensity, polarization, coherence, etc.) as the second group of light emitters 42. The processor circuitry 34 may control the emission of the electromagnetic radiation 20 by the light source 16 by separately controlling the first group of light emitters 40 from the second group of light emitters 42. For example, the first group of light emitters 40 may be positioned to treat a first type of medical condition and the second group of light emitters 42 may be positioned to treat a second type of medical condition.

The processor circuitry 34 may control the first group of light emitters 40 and the second group of light emitters 42 based on the medical condition being treated. For example, the processor circuitry 34 may include a communication interface configured to receive an identifier of medical condition(s) 92 being treated. The processor circuitry 34 may include a memory (e.g., non-transitory computer readable medium) storing a lookup table for selecting treatment properties (e.g., light emitters, power settings, timing, etc.). The processor circuitry 34 may then cause the first and second group of light emitters 40, 42 to emit electromagnetic radiation based on the received identifier 92.

The phototherapy device 10 may be used to treat many different medical conditions. When treating medical conditions associated with the neck, the light emitters 24 may be located on the pad 18, such that when the pad 18 is placed over a throat of the patient, the light emitters 24 are preferentially located between sternocleidomastoid 76 of the patient. For example, this embodiment may be used to treat at least one of dysphagia, delayed swallow reflex, pharyngeal peristalsis abnormality (PPA), voice disorder (larynx), esophageal mucositis, pharyngeal mucositis, or dysarthria.

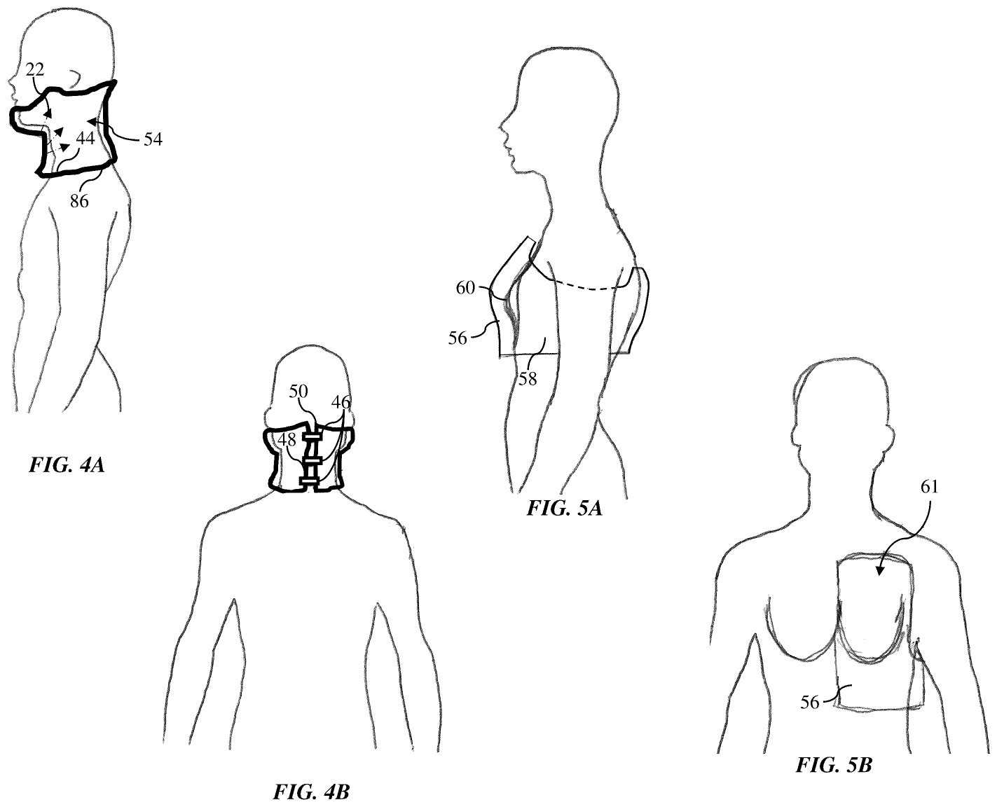

In the embodiment shown in FIG. 4, the pad 18 is contoured based on a shape of the skin surface 14, such that an inner surface (also referred to as an interior surface) 44 of the pad 18 is adjacent to the skin surface 14 when the pad 18 is pressed against the skin surface 14. The pad 18 may include a fastener 46, a proximal edge 48, and a distal edge 50. The fastener 46 may maintain a position of the distal end 50 relative to the proximal end 48, such that the inner surface 44 of the pad 18 forms a channel 54 shaped to receive at least a portion of a throat of the patient.

In the embodiment shown in FIGS. 5A and 5B, the pad 18 includes an inner surface 44, a front portion 56, and a side portion 58. The inner surface 44 of the front portion 56 has a concave portion forming a depression 60 for receiving a breast of the patient. The side portion 58 may be angled relative to the front portion 56 and may be sufficiently rigid such that the patient may maintain the position of the pad 18 by lowering their arm over the pad. That is, when the front portion 56 is positioned adjacent to the breast on a side of the patient and an adjacent arm on the side of the patient is raised, the side portion 58 of the pad 18 is located beneath the adjacent arm of the patient. Also, when the adjacent arm of the patient is lowered and pressed against a side of the patient, the position of the front portion 56 as adjacent to the breast of the patient is maintained.

The pad 18 may be shaped and sized to cover from at least an inframammary fold (underneath the breast) of the patient to an axillary fold (armpit) of the patient. The pad 18 may be flexible such that the pad takes the shape of the breast when pressed against the chest of the patient.

In one embodiment, the pad 18 may be reversible (i.e., for treating both the left and right side of the patient) and cover a single breast at a time. In another embodiment, the pad may cover both breasts of the patient simultaneously.

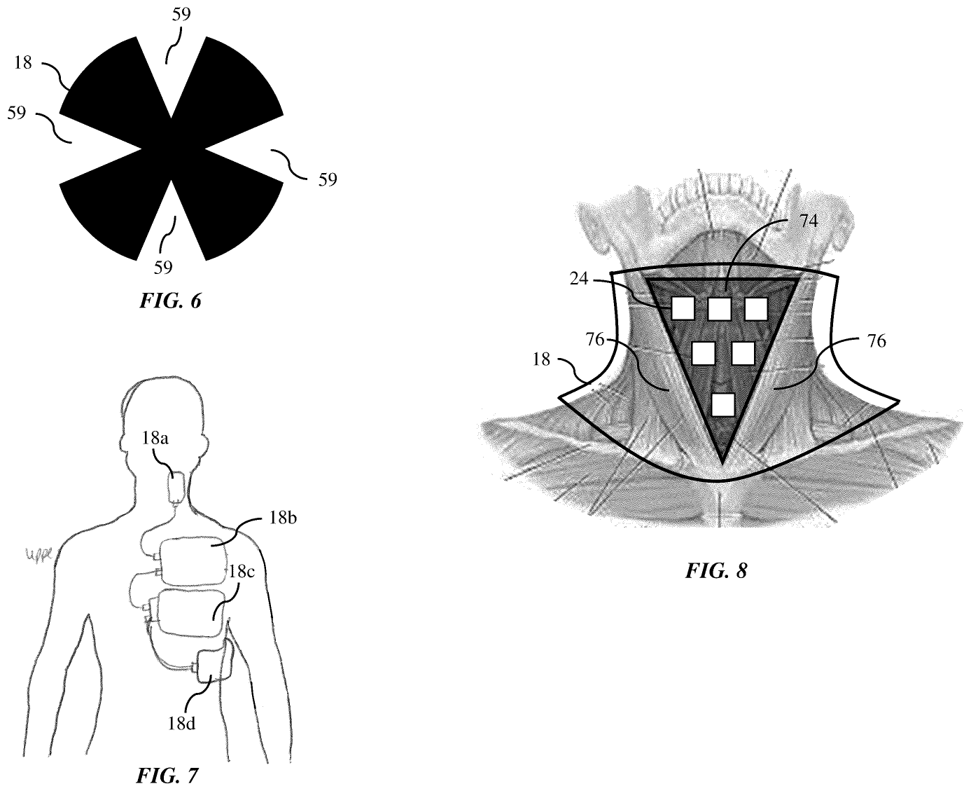

In the embodiment shown in FIG. 6, the pad includes slits 59 for use with different sized breasts. For example, for larger breasts the pad 18 will be more spread out, increasing the size of the slits 59. In the embodiment shown in FIG. 5B, the pad is shaped to cover an area of the chest above the breast (e.g., covering lymph nodes) 61.

In the embodiment shown in FIG. 7, the pad 18 comprises multiple pads 18a, 18b, 18c, 18d and each of the multiple pads 18 includes at least one of the light emitters 24 and a fastener 46 configured to maintain a position of at least one of the multiple pads 18 relative to the skin surface 14. The multiple pads 18 may share a common power source and/or processor circuitry 34. For example, the multiple pads 18 may be wired together as shown in FIG. 7.

To treat radiation dermatitis the pad 18 may be configured to accommodate multiple regions around the neck depending on the radiation field received by the patient during cancer treatment. Similarly, to treat swallowing disorders (such as dysphagia or delayed swallow reflex), electromagnetic radiation may be emitted to treat the top of the neck and under the chin. Dysphagia is defined as any subjective or objective patient complaint of trouble swallowing, coughing, choking or inability to safely handle food or secretions. A delayed swallow reflex is of particular concern when swallowing liquids because, given their low viscosity, thin liquids can easily flow downward into the larynx and trachea (leading to aspiration pneumonia, death, etc.).

In the embodiment shown in FIG. 2, the pad 18 is shaped to span from a chin 62 of the patient, around a lip 64 and nose 66 of the patient, and up to a cheekbone 68 and temporomandibular joint 70 of the patient, and down along a jawline 72 of the patient. The light emitters 24 may be located on the pad 18, such that when the pad 18 is placed on a cheek of the patient, the light emitters 24 are preferentially located along the jawline 72 relative to a cheek 68 of the patient. The light emitters 24 located along the jawline 72 may preferentially emit the treatment electromagnetic radiation, such that the optical intensity of the electromagnetic radiation emitted by the light emitters 24 located along the jawline 72 is higher than the optical intensity of the electromagnetic radiation emitted by the light emitters not located along the jawline 72.

In one embodiment, the pad is shaped to be located on only one side of the patient's face. In another embodiment, the pad may be shaped to be located on the left and right side of the patient's face. For example, the pad may be symmetrical about an axis.

The phototherapy device may be used to treat at least one of radiation dermatitis, xerostomia, radiation fibrosis, dysarthria, neuralgia/dysesthesia, osteoradionecrosis of the jaw, or recovering from jaw surgery (e.g., excising a tumor).

The phototherapy device may be used to preferentially emit treatment electromagnetic radiation 22 via light emitters 24 illuminating the “V” region (represented by the gray triangle in FIG. 8) between the sternocleidomastoid muscles, sternum, and under the chin to target the windpipe and throat region(s). In the embodiment shown in FIG. 8, the light emitters 24 are located on the pad 18, such that when the pad 18 is placed over a throat 74 of the patient, the light emitters 24 are preferentially located between sternocleidomastoid muscles 76 of the patient. The light emitters 24 located between sternocleidomastoid muscles 76 of the patient may preferentially emit the treatment electromagnetic radiation 22, such that the optical intensity of the electromagnetic radiation 20 emitted by the light emitters 24 located between sternocleidomastoid muscles 74 of the patient is higher than the optical intensity of the electromagnetic radiation 20 emitted by the light emitters 24 not located between sternocleidomastoid muscles 74 of the patient.

For example, when treatment electromagnetic radiation 22 is emitted by light emitters 24 located along a side of the neck, the treatment electromagnetic radiation 22 does not need to pass through the pharynx/air pipe, air, and throat/larynx/epiglottis in series. Instead, by coming from the side, the treatment electromagnetic radiation 22 is able to go over/under the thyroid and muscles.

When illuminating the neck, fatty tissue, and skin conditions (e.g., melanin concentrations, hair, etc.) may reduce penetration of the treatment electromagnetic radiation 22. The processor circuitry 34 may modulate a property of the treatment electromagnetic radiation 22 to mitigate these issues (e.g., using longer wavelengths). In one embodiment, a patient's fatty tissue may be physically moved to improve penetration of the treatment electromagnetic radiation. For example, the patient's fatty tissue may be moved and held in place using straps, adhesives (e.g., surgical tape). When targeting the esophagus or other centrally located structure in the neck, the treatment electromagnetic radiation may be emitted using light emitters located near the sternocleidomastoid muscles. For example, light emitters may be concentrated near the sternocleidomastoid muscles.

In one embodiment, the phototherapy device illuminates the throat/esophagus by directing light from under the chin towards the throat/esophagus. For example, the phototherapy device may treat esophageal mucositis in this manner. Esophageal mucositis is characterized by reduction in the thickness of epithelial layer, upregulation of proinflammatory cytokines and chemokines, infiltration of inflammatory cells into the esophageal mucosa, and apoptosis of epithelial cells.

In the embodiment shown in FIGS. 3A and 3B, the pad 18 includes air passages 80 permitting airflow across at least a portion of the skin surface 82. The air passages 80 may include contours 83 on an inner surface 44 of the pad 18 such that the inner surface 44 of the pad 18 does not make physical contact with the at least a portion of the skin surface 82. The air passages may also include pathways 84 between the inner surface 44 of the pad 18 and an exterior surface 86 of the pad 18 located opposite the inner surface 44 of the pad 18.

As shown in FIGS. 3B and 3C, the phototherapy device 10 may include a printed circuit board (PCB) (e.g., a flexible PCB) 85 supporting and providing electrical power to the light emitters 24. FIG. 3B is a side view of the embodiment of the phototherapy device 10 shown in FIG. 3A.

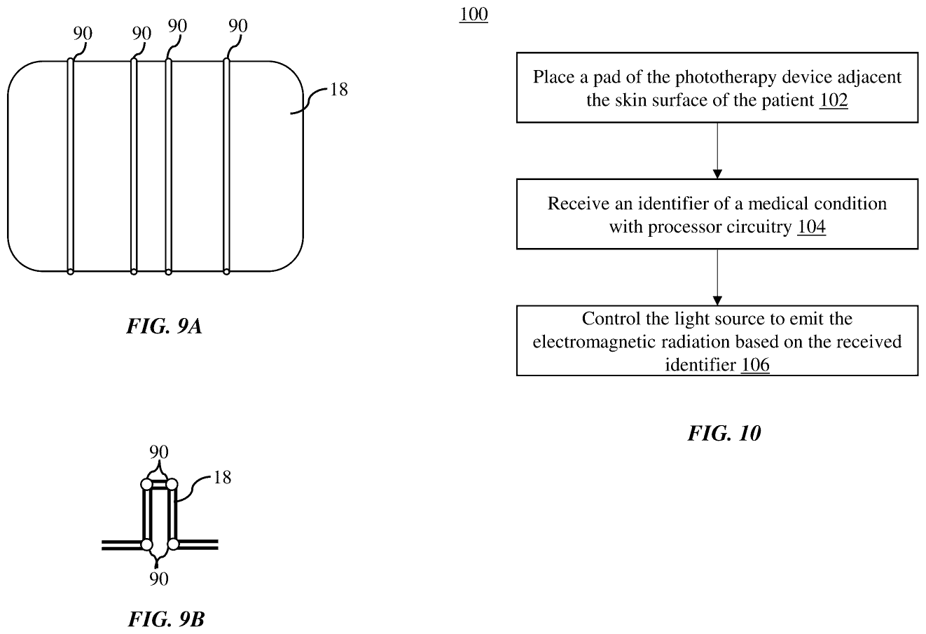

In the embodiment shown in FIGS. 9A and 9B, the pad 18 includes articulating structures 90 enabling the pad 18 to bend at a 90° (degree) angle. The articulating structures 90 may take any form that allows the pad 18 to bend more easily. For example, the articulating structures 92 may include thinner areas of the pad, hinges, etc.

The light emitters 24 may include at least one of a light emitting diode (LED), a laser diode, a light emitting end of a fiber optic, or a microLED. The light source may emit any suitable wavelength of electromagnetic radiation. The light source 16 may emit light having a wavelength from 600 nm to 1000 nm. For example, the light source 16 may emit electromagnetic radiation having a wavelength approximately equal to at least one of 630 nm, 660 nm, 670 nm, 810 nm, or 880 nm. In one embodiment, the light source may emit both therapeutic light and infrared or near infrared light, such that penetration of the therapeutic light into tissues of the oral cavity is improved. That is, the infrared or near infrared light may improve tissue penetration of the therapeutic light.

As described above, the pad 18 mechanically supports the light source 16. In one embodiment, the pad 18 additionally receives light from an external light source 16 (i.e., not mechanically supported by the pad 18. The pad 18 may also act as a light guide by receiving at least a portion of the electromagnetic radiation 20 emitted by the light source 16 and transmitting the received electromagnetic radiation via total internal reflection to a light emitting surface of the pad, such that received electromagnetic radiation is emitted from the light emitting surface and interacts with the skin surface 14.

As shown in FIG. 3C, the light emitters 24 may be covered with or include light-extracting features configured to affect a distribution of the emitted electromagnetic radiation 20. For example, at least a portion of the light emitters 24 may be embedded in the pad 18 with a portion of the pad 18 covering the light emitters 24 that includes the light-extracting features. The light-extracting features may include at least one of microscopic interruptions, lensing features, a rippled surface of the pad 18, a varying cross-sectional area of the pad 18, or varying surface finishes along the surface of the pad configured to extract light along the length of the pad 18. The light-extracting features may be chosen such that the emitted electromagnetic radiation 20 has a selected pattern (also referred to as a distribution).

The pad 18 may be made of any suitable material and have any suitable shape. In one embodiment, the pad 18 is malleable (i.e., deformable) such that a contour of the pad 18 is customizable to contours of different skin surfaces 14 and different patients. In one embodiment, at least a portion of the pad 18 is made from a soft and/or flexible material having a shore A durometer of 60 or less and a percent elongation of greater than 100%. For example, the surface of the pad 18 may have a shore A durometer of 60 or less and a percent elongation of greater than 100%. In an embodiment, the pad 18 is made of at least one of acrylic, glass, silicone, or a polymeric material. As an example, the pad 18 may be made of different formulations of polycarbonate, polymethyl methacrylate, polystyrene, nylon, acrylonitrile butadiene styrene, polyolefin, or other biocompatible thermoplastic elastomer formulations.

In one embodiment, the pad 18 includes a thermal sensor configured to monitor a surface temperature of the pad 18 or of the skin surface 14. The processor circuitry 34 may also be configured to control emission of the electromagnetic radiation 20 by the light source 16, such that emission of electromagnetic radiation 20 by the light source 16 is reduced when the thermal sensor detects a temperature greater than a predetermined level. For example, the predetermined level may be a threshold temperature (e.g., determined to cause tissue damage, damage to a part of the phototherapy device 10, or discomfort to a patient).

The property of the electromagnetic radiation 20 that varies spatially for the treatment electromagnetic radiation 22 (e.g., controlled by the processor circuitry 34) may include at least one of: an intensity, a wavelength, a duration of emission, a coherence, time modulation of emission, or a distance of emission from the target regions.

The pad 18 may include sensors positioned to detect abnormalities in the skin surface 14 (e.g., sores, differences in pigmentation, etc.). For example, the processor circuitry 34 may determine a position of one or more targeted areas based on abnormalities detected by the sensors. The processor circuitry 34 may then modulate electromagnetic radiation 20 emission by the light 16 source based on the position of the detected abnormalities, such that the detected position is preferentially illuminated compared to other locations. In another example, the phototherapy device 10 may avoid illuminating areas of detected hyperpigmentation (e.g., freckles, moles, etc.). In this example, detected areas of hyperpigmentation may receive less of an optical dose than other areas of the skin surface 14. The sensors may be photosensors configured to identify abnormalities based on visual properties (e.g., color, hue, etc.).

The phototherapy device 10 may additionally include a power source. The power source may comprise a battery and/or a plug for connecting to an external source of electricity (e.g., an electrical outlet). For example, the phototherapy device 10 may include a battery configured to provide electrical power to at the light source 16 and/or processor circuitry 34. The power source may be supported by the pad or may be located externally such that the power source is not supported by the pad.

In the embodiment shown in FIG. 10, a method 100 is shown for treating a medical condition by delivering phototherapy to an anatomical treatment area of a patient via a skin surface 14 of the patient. In step 102, a phototherapy device 10 including a pad 18 supporting a light source 16 is placed adjacent the skin surface 14 of the patient. In step 104, an identifier of the medical condition 92 is received by processor circuitry 34 of the phototherapy device. In step 106, the processor circuitry 34 causes the light source 16 to emit electromagnetic radiation 20 as treatment electromagnetic radiation 22 based on the received identifier of the medical condition 92. The treatment electromagnetic radiation 22 is controlled such that a treatment property of the treatment electromagnetic radiation 22 varies spatially based on medical properties of the anatomical treatment area 12. In one embodiment, the processor circuitry 34 determines the medical properties of the anatomical treatment area 12 based on the received identifier of the medical condition 92.

In one embodiment, the phototherapy device 10 may be used to treat medical conditions primarily affecting patients undergoing chemoradiotherapy as treatment for head and neck cancer, such as radiation dermatitis, lymphedema (neck), radiation fibrosis, neuralgia/dysesthesia, and surgery (e.g., neck flaps).

In one embodiment, the phototherapy device 10 may be used to treat lymphedema. Lymphedema (e.g., head and neck cancers, and breast cancers) is an enlargement of the lymph nodes with fluid. In this embodiment, the pad may emit therapeutic electromagnetic radiation preferentially targeting the lateral neck (e.g., this area often has the highest number of affected lymph nodes). Because the lymphatic system is closer to the surface, more shallowly penetrating therapeutic electromagnetic radiation may be emitted by the phototherapy device 10. For example, the therapeutic light may have a shorter wavelength range (such as 630 nm-660 nm, 630 nm-670, etc.).

In one embodiment, the phototherapy device 10 may be used to treat radiation fibrosis. Radiation Fibrosis following radiotherapy is characterized by increased collagen deposition (from increased differentiation of fibroblasts to myofibroblasts), poor vascularization, and scarring. In this embodiment, the pad may emit therapeutic electromagnetic radiation from light emitters targeting the sternocleidomastoid and other neck rotation muscles. The light source may also be controlled such that the therapeutic electromagnetic radiation is directed towards the skin tissue in the direct path of radiation that the patient experienced during radiation therapy. More shallowly penetrating therapeutic electromagnetic radiation may be emitted by the phototherapy device 10 when treating radiation fibrosis as well. For example, the therapeutic light may have a shorter wavelength range (such as 660 nm).

In one embodiment, the phototherapy device 10 may be used to treat neuralgia/dysesthesia. Neuralgia/dysesthesia is a chemotherapy-induced neuropathic pain. The phototherapy device may use red light (660 nm) to treat neuralgia/dysesthesia. In this embodiment, the phototherapy device may emit therapeutic electromagnetic radiation broadly targeting the neck. For example, the therapeutic electromagnetic radiation may have a shorter wavelength range (such as 660 nm).

In one embodiment, the phototherapy device 10 may be used to treat surgically caused neck flaps by illuminating the surgical location of the flap (e.g., using deeper penetrating light emitters).

In one embodiment, the phototherapy device 10 may be used to treat radiation dermatitis by generally targeting the cheek area. For example, using treatment electromagnetic radiation having a wavelength including 700 nm.

In one embodiment, the phototherapy device 10 may be used to treat xerostomia by preferentially illuminating (e.g., using a cluster of shallow penetrating light emitters) from the peak of the cheek bone down to the corner of the jaw and including the parotid gland and/or submandibular gland. Xerostomia or Dry Mouth can be a side effect of chemotherapy and radiation therapy to the head and neck. Xerostomia results from inadequate function of the salivary glands (the parotid, submandibular and sublingual glands). The phototherapy device 10 may also include light emitters located submandibular and directing therapeutic light from under the mandible/under chin (e.g., using deeper penetrating light emitters).

In one embodiment, the phototherapy device 10 may be used to treat radiation fibrosis. For example, the phototherapy device may illuminate the general cheek area. The phototherapy device may preferentially illuminate the facial muscles used to talk and chew (which are often affected by radiation fibrosis).

In one embodiment, the phototherapy device 10 may be used to treat dysarthria by illuminating at least one of the masseter, buccinator, depressor labii, depressor anguli oris muscles. A person with dysarthria may have problems controlling the pitch, loudness, rhythm, and voice qualities of his or her speech. Dysarthria is caused by paralysis, weakness, or inability to coordinate the muscles of the mouth. The phototherapy device may utilize treatment electromagnetic radiation having a longer wavelength and a higher intensity to reach deeper tissues.

In one embodiment, the phototherapy device 10 may be used to treat neuralgia/dysesthesia by generally illuminating the cheek.

In one embodiment, the phototherapy device 10 may be used to treat osteonecrosis (also referred to as osteoradionecrosis) of the jaw (e.g., caused by mandible bone deterioration) or to improve patient healing following jaw surgery. Osteoradionecrosis is a problematic complication that occurs when irradiated bones become devitalized. In this embodiment, the phototherapy device may preferentially generate the treatment electromagnetic radiation using light emitters located along a line extending from the temporomandibular joint downwards and thickening as you get to the chin. The phototherapy device may similarly use deeper penetrating light emitters (e.g., including the wavelength 850 nm or greater, 660 nm at a greater optical intensity, etc.) to reach bone tissue.

In one embodiment, the phototherapy device 10 may be used to treat radiation dermatitis of the breast and/or chest wall (e.g., depending on location of cancer or medical condition caused by cancer treatment). For example, radiation dermatitis is often worse in the inframammary fold and the axillary fold, but radiation dermatitis is not limited to these regions. The phototherapy device may treat radiation dermatitis by illuminating affected areas using therapeutic electromagnetic radiation having a shorter wavelength range (such as 660 nm).

In one embodiment, the phototherapy device 10 may be used to treat lymphedema (e.g., post mastectomy). The phototherapy device may illuminate areas known to have a high concentration of lymph nodes (e.g., lateral chest wall, under and in armpit, etc.).

The phototherapy device may include an electrical power storage device such as a battery. Alternatively or additionally, the phototherapy device may receive electrical power from an external power source (e.g., plugging into a wall) or a battery not mechanically supported by the pad (e.g., a separate battery pack).

In one embodiment, the phototherapy device includes multiple pads. The pads may be separately controllable. Alternatively, the pads may be electrically connected to share electrical power. The pads may also be separately controllable by processor circuitry located on each pad or processor circuitry located on a subset (e.g., one) of the pads.

The pads may include light-extracting features (e.g., lenses) to scatter and/or focus light over a large surface. Ridge like features also prevent line-to-line contact with skin, to allow for more breathability.

The phototherapy device may additionally include a support configured to maintain a position of the pad relative to the patient. For example, the support may be a harness and/or straps for securing the pads relative to the patient. The support may also be an adhesive such as surgical tape.

The processor circuitry 34 may have various implementations. For example, the processor circuitry 34 may include any suitable device, such as a processor (e.g., CPU), programmable circuit, integrated circuit, memory and I/O circuits, an application specific integrated circuit, microcontroller, complex programmable logic device, other programmable circuits, or the like. The processor circuitry 34 may also include a non-transitory computer readable medium, such as random-access memory (RAM), a read-only memory (ROM), an erasable programmable read-only memory (EEPROM or Flash memory), or any other suitable medium. Instructions for performing the method described below may be stored in the non-transitory computer readable medium and executed by the processor circuitry 34. The processor circuitry 34 may be communicatively coupled to the computer readable medium and a network interface through a system bus, mother board, or using any other suitable structure known in the art. The processor circuitry 34 may receive parameters for controlling the light source 16 via the network interface.

All ranges and ratio limits disclosed in the specification and claims may be combined in any manner. Unless specifically stated otherwise, references to “a,” “an,” and/or “the” may include one or more than one, and that reference to an item in the singular may also include the item in the plural.

Although the invention has been shown and described with respect to a certain embodiment or embodiments, equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

1. A phototherapy device for treating a medical condition by delivering phototherapy to an anatomical treatment area of a patient via a skin surface of the patient, the system comprising:
a light source configured to emit electromagnetic radiation; and
a pad configured to support the light source, such that positioning the pad adjacent to the skin surface causes the electromagnetic radiation output by the light source to be received by the skin surface as treatment electromagnetic radiation and to interact with the anatomical treatment area;
wherein the light source includes multiple light emitters located at different positions on the pad, such that a treatment property of the treatment electromagnetic radiation varies spatially based on medical properties of the anatomical treatment area;
wherein the light emitters are located on the pad, such that when the pad is placed over a throat of the patient, the light emitters are preferentially located between sternocleidomastoid muscles of the patient; and
wherein the medical properties include at least one of anatomical structures or the medical condition.

2. The phototherapy device of claim 1, wherein the treatment property that varies spatially comprises at least one of wavelength, optical intensity, or optical dose.

3. The phototherapy device of claim 1 or 2, wherein:
the light emitters include at least one first type of light emitter and at least one second type of light emitter;
the treatment property of the electromagnetic radiation emitted by the first type of light emitter differs from the treatment property of the electromagnetic radiation emitted by the second type of light emitter; and
at least one of a total number or a position of the at least one first type of light emitter differs from that of the at least one second type of light emitter, such that the treatment property of the treatment electromagnetic radiation varies spatially.

4. The phototherapy device of claim 3, wherein:
the at least one first type of light emitter is configured to emit a first wavelength range of electromagnetic radiation;
the at least one second type of light emitter is configured to emit a second wavelength range of electromagnetic radiation; and
the first wavelength range differs from the second wavelength range.

5. The phototherapy device of claim 3, further comprising processor circuitry configured to control the emission of the electromagnetic radiation by the light source by separately controlling the first type of light emitters from the second type of light emitters.

6. The phototherapy device of claim 5, wherein the processor circuitry is configured to control the first type of light emitters and the second type of light emitters based on the medical condition being treated.

7. The phototherapy device of claim 1, wherein a positioning of the multiple light emitters varies spatially, such that the optical dose of the treatment electromagnetic radiation varies spatially.

8. The phototherapy device of claim 1:
wherein the light emitters include at least a first group of light emitters and a second group of light emitters;
further comprising processor circuitry configured to control the emission of the electromagnetic radiation by the light source by separately controlling the first group of light emitters from the second group of light emitters.

9. The phototherapy device of claim 8, wherein:
the first group of light emitters are positioned to treat a first type of medical condition; and
the second group of light emitters are positioned to treat a second type of medical condition.

10. The phototherapy device of claim 9, wherein the processor circuitry is configured to control the first group of light emitters and the second group of light emitters based on the medical condition being treated.

11. The phototherapy device of claim 1, wherein the pad is contoured based on a shape of the skin surface, such that an inner surface of the pad is adjacent to the skin surface when the pad is pressed against the skin surface.

12. The phototherapy device of claim 11, wherein:
the pad includes a fastener, a proximal edge, a distal edge, and an inner surface;
the fastener is configured to maintain a position of the distal end relative to the proximal end, such that the inner surface of the pad forms a channel shaped to receive at least a portion of a throat of the patient.

13. (canceled)

14. The phototherapy device of claim 1:
wherein the pad comprises multiple pads and each of the multiple pads includes at least one of the multiple light emitters; and
further comprising a fastener configured to maintain a position of at least one of the multiple pads relative to the skin surface.

15. (canceled)

16. (canceled)

17. The phototherapy device of claim 1, wherein:
the light emitters located between sternocleidomastoid muscles of the patient preferentially emit the treatment electromagnetic radiation, such that the optical intensity of the electromagnetic radiation emitted by the light emitters located between sternocleidomastoid muscles of the patient is higher than the optical intensity of the electromagnetic radiation emitted by the light emitters not located between sternocleidomastoid muscles of the patient.

18. The phototherapy device of claim 1, wherein:
the pad includes air passages configured to permit airflow across at least a portion of the skin surface; and
the air passages include at least one of:
contours on an inner surface of the pad such that the inner surface of the pad does not make physical contact with the at least a portion of the skin surface; or
passages between the inner surface of the pad and an exterior surface of the pad located opposite the inner surface of the pad.

19. The phototherapy device of claim 1, wherein the pad includes articulating structures configured to enable the pad to bend at a 90-degree angle.

20. The phototherapy device of claim 1, wherein each of the multiple light emitters is at least one of a light emitting diode (LED) or a laser diode.

21. The phototherapy device of claim 1, wherein the pad acts as a light guide by receiving at least a portion of the electromagnetic radiation emitted by the light source and transmitting the received electromagnetic radiation via total internal reflection to a light emitting surface of the pad, such that received electromagnetic radiation is emitted from the light emitting surface and interacts with the tissue.

22. (canceled)

23. The phototherapy device of claim 1:
wherein the pad includes a thermal sensor configured to monitor a surface temperature of the probe; and
further comprising processor circuitry configured to control emission of the electromagnetic radiation by the light source, such that emission of electromagnetic radiation by the light source is reduced when the thermal sensor detects a temperature greater than a predetermined level.

24. The phototherapy device of claim 1, wherein the light source is configured to emit both therapeutic light and infrared or near infrared light, such that penetration of the therapeutic light into the anatomical treatment area is improved.

25. A method for treating a medical condition by using a phototherapy device to deliver phototherapy to an anatomical treatment area of a patient via a skin surface of the patient, the method comprising:
placing a pad of the phototherapy device adjacent the skin surface of the patient, such that:
electromagnetic radiation emitted by a light source of the phototherapy device is received by the skin surface as treatment electromagnetic radiation; and
the treatment electromagnetic radiation interacts with the anatomical treatment area, wherein the pad supports the light source;
receive an identifier of the medical condition with processor circuitry of the phototherapy device;
the processor circuitry controlling the light source to emit the electromagnetic radiation based on the received identifier of the medical condition, such that a treatment property of the treatment electromagnetic radiation varies spatially based on medical properties of the anatomical treatment area;
wherein the medical properties include at least one of anatomical structures or the medical condition.

26. The method of claim 17, wherein the medical condition comprises at least one of radiation dermatitis, dysphagia, delayed swallow reflex, pharyngeal peristalsis abnormality, esophageal mucositis, pharyngeal mucositis, dysarthria, lymphedema, radiation fibrosis, neuralgia, or dysesthesia.

27. The method of claim 17, wherein the medical condition comprises at least one of radiation dermatitis, xerostomia, radiation fibrosis, dysarthria, neuralgia/dysesthesia, or osteoradionecrosis of the jaw.
